(19) United States
(12) Patent Application Publication
(10) Pub. No.: US 2013/0046262 A1
(43) Pub. Date: Feb. 21, 2013

(54) RENEWABLE THERMOPLASTIC STARCH-BASED MULTI-LAYER FILMS AND ARTICLES

(71) Applicants: James H. Wang, Appleton, WI (US); Brent M. Thompson, Oshkosh, WI (US); William Laratta, Curridabat (CR); Marcelo P. Paulino, Sapucaia do Sul - RS (BR)

(72) Inventors: James H. Wang, Appleton, WI (US); Brent M. Thompson, Oshkosh, WI (US); William Laratta, Curridabat (CR); Marcelo P. Paulino, Sapucaia do Sul - RS (BR)

(21) Appl. No.: 13/219,984

(22) Filed: Aug. 29, 2011

Related U.S. Application Data
(63) Continuation-in-part of application No. 13/211,572, filed on Aug. 17, 2011.

(51) Int. Cl. A61L 15/22; B32B 7/02; B32B 5/00; B32B 3/00; B32B 27/08; B32B 27/32

(52) U.S. Cl. 604/370; 428/515; 428/516; 428/220; 428/141; 428/216

(57) ABSTRACT

The present invention relates to a multiple layer polymeric film comprising at least three layers wherein at least two layers comprise at least one polyolefin and a third layer comprises from about 5% to about 45% of a thermoplastic starch, from about 55% to about 95% of at least one polyolefin, and from about 0.5% to about 10% of a compatibilizer, wherein said compatibilizer is selected from the group consisting of a graft copolymer, a block copolymer, and a random copolymer of non-polar monomers and polar monomers. Also presented is a packaging material or a consumer product comprising a portion made of the multiple layer polymeric film that may be used to create an absorbent article such as diapers, pantiliners, feminine pads, adult incontinence products, wipes, tissues, and the like.

### CLAIM OF BENEFIT OF PRIORITY

The present application is a continuation-in-part and claims benefit of priority to U.S. patent application Ser. No. 13/211,572, filed on Aug. 17, 2011, the contents of which are incorporated herein.

### FIELD OF INVENTION

The present invention relates to a composition for flexible polyolefin-based films that contain thermoplastic starches. In particular, the invention pertains to packaging films that include polyolefins, renewable polymers, and a compatibilizer, and describes a method to overcome their material incompatibility to make packaging films of desirable physical and mechanical properties.

### BACKGROUND

In recent years as petroleum resources have become more scarce or expensive and manufacturers and consumers alike have become more aware of the need for environmental sustainability, interest in biodegradable and renewable films containing renewable and or natural polymers for a variety of uses has grown. Renewable polymers available today, such as polylactic acid (PLA), polyhydroxyalkanoate (PHA), thermoplastic starch (also referred to herein as “TPS”) and the like, however, all have deficiencies in making thin, flexible packaging films such as those that are typically used as packaging films for bath tissues, facial tissue, wet wipes and other consumer tissue products, product bags for personal care products, away-from-home products, and health care products. For instance, PLA thin film exhibits a high stiffness and very low ductility, sometimes costly bi-axial stretching process is used to produce thin PLA films, which results in relatively high “rustling” noise levels when handled and very stiff films, making the material unsuitable for flexible thin film packaging uses. PHA is difficult to make into thin films. Poor film processability (i.e., slow crystallization, “extreme” stickiness prior to solidification) retards fabrication-line speeds and results in relatively expensive production costs. Some PHA such as poly-3-hydroxybutyrate (PHB), poly-3-hydroxybutyrate-co-3-hydroxyvalerate (PHBV) films have high stiffness and low ductility, making them unsuitable for flexible thin film applications. When used alone as a film, thermoplastic starch has a low tensile strength, low ductility, and also severe moisture sensitivity. Due to its low melt strength and extensibility, thermoplastic starch has been unsuitable for stand-alone packaging film applications unless blended with an expensive biodegradable polymer such as Ecoflex™, an aliphatic-aromatic copolyester by BASF AG.

Typical existing packaging equipment are optimal for converting polyethylene (also referred herein as “PE”)-based films, efforts to replace or upgrade the packaging hardware to run 100% renewable polymers is likely to require high capital expenditures. The poor processability of 100% renewable polymers also increases production cost due to reduced line speed, etc. Therefore, there is a need for thin packaging films containing a renewable polymer to reduce the carbon footprint and improve environmental benefits at an affordable cost. The packaging films must have good performance required for packaging applications in terms of heat seal, tensile properties, and free of any visible defects, and suitability for high speed packaging applications.

### SUMMARY OF THE INVENTION

The present invention relates to a multiple layer polymeric film comprising at least three layers wherein at least two layers comprise at least one polyolefin and the third layer comprises from about 5% to about 45% of a thermoplastic starch, from about 55% to about 95% of at least one polyolefin, and from about 0.5% to about 10% of a compatibilizer, wherein said compatibilizer is selected from the group consisting of graft copolymers, block copolymers, and random copolymers of non-polar and polar monomers.

The present invention also relates to a flexible multiple layer polymeric film comprising from about 5% to about 55% of a thermoplastic starch masterbatch and from about 40% to about 95% of a polyolefin or mixtures of polyolefins.

Further, the present invention relates to a packaging material or a consumer product comprising a portion made from the multiple layer polymeric film of the present invention. The consumer product may be an absorbent article such as diapers, pantiliners, feminine pads, adult incontinence products, wipes, tissues, and the like.

### BRIEF DESCRIPTION OF FIGURES

FIG. 1 is a representation of the molecular structure of Amylopectin.

FIG. 2 is a representation of the molecular structure of Amylose.

FIG. 3 shows a photo of a comparative example of a film formed from a blend of 80% polyethylene and 20% TPS, having undispersed TPS aggregates (white dots) and holes that have developed due to the stretching in the machine direction.

FIG. 4 shows a photo of another comparative example of a film similar to that of FIG. 3. The film has 30% TPS blended with 70% polyethylene, exhibiting a greater number of undispersed starch aggregates and large holes in the film.

FIG. 5 is the molecular structure of a maleic anhydride grafted copolymer of a polyolefin (DuPont Fusabond® MB-528D).

FIG. 6 shows a photo of an example of a film according to the present invention containing 10% TPS, 90% polyethylene, and 1% compatibilizers. The undispersed TPS that was previously seen in the films of FIGS. 3 and 4 are nonexistent in this example of the film composition.

FIG. 7 shows another example of a film according to the present invention containing 40% TPS, 60% polyethylene, and 5% compatibilizer. Similar to FIG. 6, the film exhibits little evidence of undispersed starch aggregates and no holes. The starch was fully homogenized up to about 40-45%.

FIG. 8 is a graph that shows the dispersion region for relative incorporated amounts of compatibilizer as a function of the polyolefin content in several different blends of PE and TPS.

FIG. 9 is a graph of the moduli of five film samples with different levels of TPS incorporation.

FIG. 10 is a graph that summarizes the peak stress of the five films of FIG. 9.

FIG. 11 is a graph that summarizes the strain-at-break of the five films of FIGS. 9 and 10.

FIG. 12 is a graph that presents the energy-to-break of film samples according to the invention, along machine direction (MD) and cross-direction (CD) stretching.

FIG. 13 is a graph that presents the moduli of four 60% PE, 40% TPS films that were blended with different percentage amounts of compatibilizer (Fusabond® MB-528D).

FIG. 14 is a graph that shows the peak stress of the same four blends of FIG. 13.

FIG. 15 is a graph that shows the strain-at-break of the four blends of FIG. 13.

FIG. 16 is a graph that shows the energy-to-break of the films made from the four blends of FIG. 13.

FIG. 17 shows an example of a multi-layer film wherein three layers are shown.

### DETAILED DESCRIPTION OF THE INVENTION

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein. Except where specific examples of actual measured values are presented, numerical values referred to herein should be considered to be qualified by the word “about”.

As used herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

While the specification concludes with the claims particularly pointing out and distinctly claiming the invention, it is believed that the present invention will be better understood from the following description.

The term “biodegradable,” as used herein, refers generally to a material that can degrade from the action of naturally occurring microorganisms, such as bacteria, fungi, yeasts, and algae; or environmental heat, moisture, or other environmental factors. If desired, the extent of biodegradability may be determined according to ASTM Test Method 5338.92.

“Energy-to-break” refers to the total area under the stress vs. strength curve.

The term “modified starch” as used herein, refers to starches which have been modified chemically or enzymatically by the typical processes known in the art (e.g., esterification, etherification, oxidation, acidic hydrolysis, enzymatic hydrolysis, crosslinking, carboxymethylation, etc.). Typical modified starches are starch ethers (e.g. methyl starch, ethyl starch, propyl starch, etc.), esters (e.g. starch acetate, starch propionate, starch butyrate, etc.), hydroxyalkyl starches (hydroxymethyl starch hydroxyethyl starch, hydroxypropyl starch, etc.); carboxymethyl starches, etc.

“Modulus” refers to the slope of the initial portion of the stress vs. strength curve.

The term “native starch” as used herein, refers to unmodified starch separated from plants, typical sources includes seeds of cereal grains, such as corn, waxy corn, wheat, sorghum, rice, and waxy rice; tubers, such as potatoes; roots, such as tapioca, (i.e. cassava and manioc), sweet potatoes, and arrowroot; and the pith of the sago palm.

“Peak stress” refers to the value of stress level at peak.

The term “renewable” as used herein refers to a material that can be produced or is derivable from a natural source which is periodically (e.g., annually or perennially) replenished through the actions of plants of terrestrial, aquatic or oceanic ecosystems (e.g., agricultural crops, edible and non-edible grasses, forest products, seaweed, or algae), or microorganisms (e.g., bacteria, fungi, or yeast).

“Strength-at-break” refers to the strength value when the sample breaks.

In general, the invention describes a flexible polymeric film having from about 5% to about 45% of a thermoplastic starch, from about 55% to about 95% of a polyolefin or mixtures of polyolefins, and from about 0.5% to about 10% of a compatibilizer, which is a graft copolymer of a non-polar backbone and a grafted polar monomer, or a block copolymer of both a non-polar block and a polar block, or a random copolymer of a non-polar monomer and a polar monomer. The amounts of said thermoplastic starch and compatibilizer, respectively, can be present in a ratio of between about 2.5:1 to about 95:1. Typically, the ratio of said thermoplastic starch and compatibilizer, respectively, is between about 5:1 and about 55:1. More typically, the ratio of said thermoplastic starch and compatibilizer, respectively, is between about 10:1 and about 30:1.

The invention relates, in part, to a method of forming a polymeric film, the method comprising: preparing a polyolefin mixture, blending said polyolefin mixture with a thermoplastic starch and a compatibilizer, which is a graft copolymer having a non-polar backbone and a grafted polar monomer or a block copolymer of both a non-polar block and a polar block or a random copolymer of a non-polar monomer and a polar monomer, said thermoplastic starch and compatibilizer, respectively, are present in amounts in a ratio of between about 2.5:1 to about 95:1; extruding said film of said blended polyolefin mixture.

In another aspect the present invention pertains to a packaging material or assembly made from the polymeric film composition such as described. The film can be fabricated to be part of a packaging assembly. The packaging assembly can be used to wrap consumer products, such as absorbent articles including diapers, adult incontinence products, pantiliners, feminine hygiene pads, or tissues. In other iterations, the invention relates to a consumer product having a portion made using a flexible polymeric film, such as described. The polymeric film can be incorporated as part of consumer products, e.g., baffle films for adult and feminine care pads and liners, outer cover of diapers or training pants, and the like.

Additional features and advantages of the present invention will be revealed in the following detailed description. Both the foregoing summary and the following detailed description and examples are merely representative of the invention, and are intended to provide an overview for understanding the invention as claimed.

The present invention addresses a need for a flexible polymeric film that is better or improved over conventional polyolefin films in terms of its environmental impact. The use of renewable materials in films containing natural or new carbon, or recently fixed CO₂, can slightly reduce global warming effects. The production of the present inventive films can reduce energy input and greenhouse gas emissions. The relative degree of biodegradation somewhat depends on the amount of biodegradable component present in the films, but it is more biodegradable than pure polyolefin thin films.

Additionally, the present invention enables packaging manufacturers to make use of a majority of polyolefins and a minority of renewable materials to achieve good processing characteristics and mechanical properties at low cost. The present invention describes a composition for and method of making thin packaging films for consumer packaged goods and products with suitable performance, renewable polymer content to reduce their environmental footprint, and at an attractive cost. The composition incorporates renewable polymers such as thermoplastic starch as a renewable component. The amount of renewable polymers has to be at a volumetric minority so the polyolefin properties will dominate the blend properties. An appropriate type of compatibilizer at the right amount must be employed to compatibilize the hydrophobic polyolefin(s) phase and hydrophilic thermoplastic starch phase to create an adequate dispersion and good film properties.

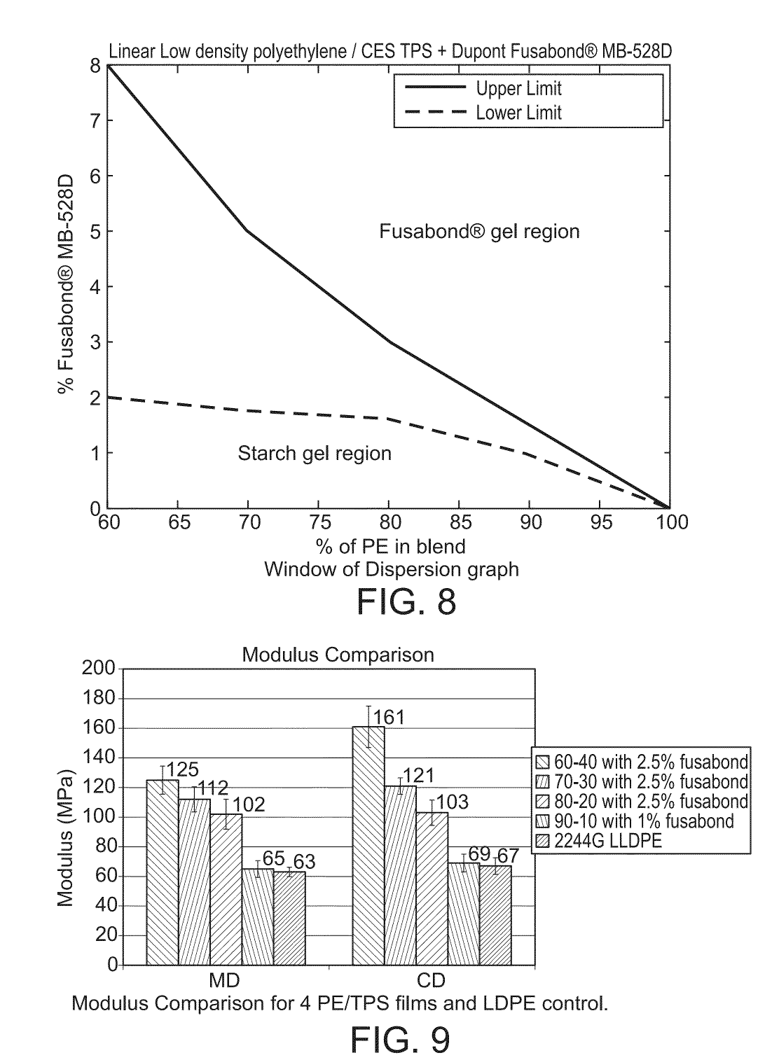

It was surprisingly found that a range and ratio of thermoplastic starch, polyolefin and compatibilizer allows the blends to have good physical and mechanical properties. At a particular range and ratio, compositions of the present invention were found to have good mechanical properties, good processability, and to be free from any visible defects. As shown in FIG. 8, compositions that fell outside of the particular ranges found and disclosed in the present invention formed gelled phases of either thermoplastic starch or compatibilizer that resulted in poor mechanical properties, visual defects, and made the films unsuitable for packaging applications. For compositions with too little compatibilizer, the renewable polymers such as thermoplastic starch formed un-dispersed gels leading to granular defects and visible voids/holes that made it unsuitable for thin packaging film applications. In compositions with an above optimal range of compatibilizer, the compatibilizer formed a gelled phase and produced defects. Another aspect of this invention is that the polyolefin in the film material can be processed relatively easily and achieves good tensile strength and cohesive properties that allow packaging films to be produced at no productivity penalty or slowdown in converting process. Also disclosed in this invention are multiple-layered co-extruded flexible packaging films comprising one or more layers of polyolefin or polyolefin mixture layers; the presence of one or more polyolefin layers providing excellent sealability, printability, and mechanical properties required for packaging consumer packaged goods.

In comparison to conventional polyolefin-based films, the inventive polymeric film may be much softer and more breathable to moisture. In some applications, such as absorbent articles, the film of the present invention is able to keep a user's skin drier. When the present films are employed in such articles as a baffle film in a feminine or adult care pad or the outer cover film of a diaper, training pants, or adult incontinence pants, the film will feel more comfortable against the user's skin as a consequence of a more micro-grainy or micro-textured surface, and will not have as slippery or rubbery a feeling as conventional polyethylene-based films.

The thermoplastic starch in the polymeric film comprises either a native starch or a modified starch with a plasticizer. The native starch can be selected from corn, wheat, potato, rice, tapioca, cassava, etc. The modified starch can be a starch ester, starch ether, oxidized starch, hydrolyzed starch, hydroxyalkylated starch, and the like. Genetically modified starch can also be used. Such genetically modified starch may have a different ratio from that of amylose and amylopectin than native starches. Mixtures of two or more different types of native starch or modifications thereof can also be used in this invention.

The thermoplastic starch may include a plasticizer or mixture of two or more plasticizers selected from polyhydric alcohols including glycerol, glycerine, ethylene glycol, polyethylene glycol, sorbitol, citric acid and citrate, aminoethanol, and the like. In certain embodiments, the concentration of starch in thermoplastic starch may be from about 45 wt. % or 50 wt. % to about 85 wt. % or 90 wt. %. One may include proportionate amounts of mixed starches of different origins or types (e.g., starches selected from corn, wheat, potato, rice, tapioca, cassava, etc.). According to certain other embodiments, the amount of thermoplastic starch may include from about 60 wt. % or about 65 wt. % to about 85 wt. % or about 90 wt. % starch, and from about 10 wt. % or about 15 wt. % to about 35 wt. % or about 40 wt. % plasticizers, inclusive of any combination of ranges there between.

Thermoplastic starch based biodegradable plastics of the present invention have a starch content greater than about 60% and are based on vegetable starch. With the use of specific plasticizers, such plastics can produce thermoplastic materials with good performance properties and inherent biodegradability. Starch is typically plasticized, destructured, and/or blended with other materials to form thermoplastic starch with useful mechanical properties. Importantly, such thermoplastic starch compounds can be processed on existing plastics fabrication equipment.

High starch content plastics are highly hydrophilic and may absorb moisture upon extended exposure to high humidity or upon contact with water. This can be overcome through blending with other polymers. Alternatively, as the starch has free hydroxyl groups which readily undergo a number of reactions such as acetylation, esterification and etherification, thermoplastic starch can be made from modified starch (e.g. starch ethers, esters, etc.) to reduce its water sensitivity.

The resulting flexible film includes about 5% to about 45% of a renewable polymer such as thermoplastic starch, from about 55% to about 95% of at least one polyolefin, and from about 0.5% to about 10% of a compatibilizer, wherein the compatibilizer has a graft copolymer having a non-polar backbone and a grafted polar monomer or a block copolymer of both a non-polar block and a polar block or a random copolymer of a non-polar monomer and a polar monomer.

According to alternate embodiments, the flexible polymeric film may incorporate a masterbatch or a concentrate of thermoplastic starch (“TPS masterbatch”). As used herein, “TPS masterbatch” refers to a blend of thermoplastic starch, at least one polyolefin or a mixture of polyolefins, and compatibilizers. The TPS masterbatch of the present invention may comprise from about 40% to about 90% of a thermoplastic starch, from about 10% to about 45% of a polyolefin or a mixture of polyolefins, and from about 1% to about 10% of compatibilizers, wherein said compatibilizers may be a graft copolymer of a non-polar backbone and a grafted polar monomer or a block copolymer of both a non-polar block and a polar block or a random copolymer of a non-polar monomer and a polar monomer. The mixture of polyolefins may comprise low density polyethylene, high density polyethylene, linear low density polyethylene, linear medium density polyethylene, linear ultra-low density polyethylene, polypropylene, ethylene propylene copolymers, and the like.

According to alternate embodiments of the present invention, the flexible polymeric film may incorporate a color concentrate, and a polyolefin or a mixture of polyolefins. The flexible film may comprise from about 1% to about 15% of a color concentrate. The color concentrate can be added to make the otherwise clear film opaque, or white, or other colors. Color concentrates may include, for instance, various dyes, titanium oxide, calcium carbonate, opacifiers such as clays, and the like. Alternatively, the TPS masterbatch may also comprise a color concentrate and may have from about 50% to about 90% by weight a thermoplastic starch, from about 5 to about 40% a polyolefin or a mixture of polyolefins, and from about 0.5% to about 5% a compatibilizer, and from about 1% to about 15% a color concentrate.

Examples of the polyolefins that may be incorporated include low density polyethylene (LDPE), high density polyethylene (HDPE), linear low density polyethylene (LLDPE), metallocene catalyzed polyolefins, very low density polyethylene (VLDPE), ultra-low density polyethylene (ULDPE), single site catalyzed polyethylene, polypropylene (PP), ethylene-propylene copolymers, polyolefin elastomers such as Vistmaxx® from Exxon Mobil, ethylene copolymers, polyolefin elastomers of block copolymers of ethylene and propylene, or ethylene copolymers with vinyl acetate, methacrylate, acrylic acid, methacrylic acid, and the like.

The compatibilizer may include: polyethylene-co-vinyl acetate (EVA), polyethylene-co-vinyl alcohol (EVOH), polyethylene-co-acrylic acid (EAA), polyethylene-co-methacrylic acid (EMAA), polyolefin graft copolymer of non-polar polyolefin backbone grafted with a polar monomer such as a polyethylene grafted with maleic anhydride or polypropylene grafted with maleic anhydride or polyethylene grafted with glycidyl methacrylate. The polar monomer can include maleic anhydride, acrylic acid, vinyl acetate, vinyl alcohol, vinyl amine, acrylamide, or acrylate, glycidyl acrylate, glycidyl methacrylate, and the like. The polar monomer may be present in an amount that ranges from about 0.1%, about 0.3%, about 0.5% or about 1% to about 35%, about 37%, about 40%, about 45% by weight of the composition. Mixed polyolefins or polyethylene/polypropylene blends can also be used in this invention. The composition may also contain from about 0.5% to about 30% of a biodegradable polymer.

The polymeric film can include a mineral filler that is present in an amount from about 5% or about 8% to about 33% or about 35% by weight of the composition. Typically, the mineral filler is present in an amount from about 10% or about 12% to about 25% or about 30% by weight of the composition. The mineral filler may be selected from any one or a combination of the following: talcum powder, calcium carbonate, magnesium carbonate, clay, silica, alumina, boron oxide, titanium oxide, cerium oxide, germanium oxide, diatomaceous earth (DE), and the like.

### Multi-Layer Films

The polymeric packaging films can have multiple layers, for instance, from 2 to 7 or 20 layers; or in some embodiments, from 2 or 3 to 10 layers. Each layer may have a thickness from about 0.05 mil to about 2.0 mil (1 mil=25.4 micrometers). Typically, each layer has a thickness from about 0.1 mil to about 1 mil or from about 0.2 mil to about 0.5 mil. The combined polymeric film layers can have an overall thickness from about 0.5 mil to about 5.0 mil, typically from about 0.7 mil to about 4 mil or from about 1 mil to about 2 mil. Each layer can have a different composition, but at least one of the layers is formed from the present inventive film composition. At least one layer of the present invention is formed with a TPS masterbatch. The thermoplastic starch content (“TPS content”) of a TPS masterbatch, can range from about 40% to about 90% by weight of the TPS masterbatch. In some embodiments, the TPS content may be from about 50% to about 85% of the masterbatch. The polyolefin in the layer can be low density polyethylene, linear low density polyethylene, linear medium density polyethylene, linear ultra-low density polyethylene, high density polyethylene or ethylene copolymers, polypropylene, or mixtures of polyolefins. At least one layer on the seal side of the film comprises polyolefin. As used herein, “seal side” refers to the layer of the film that is the innermost layer.

In an alternative embodiment, the outside layer of the multi-layer film may comprise at least one polyolefin or a mixture of polyolefins. Such embodiment is ideal when forming a product or a product bag such as that to package or bundle diapers. In yet another embodiment, the printing layer and the seal side layers may comprise at least one polyolefin or a mixture of polyolefins, or a mixture of polyolefins with a TPS masterbatch. As used herein, the “printing layer” refers to the outermost layer of a product or package. The mixture of polyolefins may comprise low density polyethylene, high density polyethylene, linear low density polyethylene, linear medium density polyethylene, polypropylene, and the like. The polyolefin content in these layers ranges from about 10% to about 90%, by weight of the composition and the total thermoplastic starch and compatibilizer constitute from about 10% to about 90%, by weight of the composition. In an embodiment of the present invention comprising more than three layers, at least one inside layer (not including the seal side layer) may comprise at least one polyolefin, a mixture of polyolefins or a mixture of polyolefins with a TPS masterbatch. Additionally, in an embodiment wherein there are more than three layers, at least one outer layer (not including the printing layer) may comprise at least one polyolefin or a mixture of polyolefins, or a mixture of polyolefins with a TPS masterbatch.

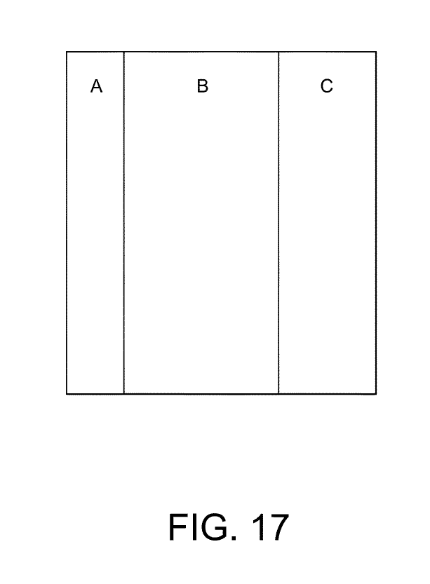

In one particular embodiment, the multi-layer film has three layers. Each of the outside layers constitutes from about 5% to about 45% of the total thickness of the three-layer film and the middle layer constitutes from about 5% to about 45% of the total layer film thickness. In one embodiment, a three layer film has a heat seal layer A with a thickness of about 20% of the overall thickness of the three-layer film, a middle layer B, which is about 55% of the total thickness, and an outside printing layer C, which is about 25% of the total film thickness (as shown in FIG. 17).

Generally, the flexible polymeric film according to the invention exhibits a modulus from about 50 MPa to about 300 MPa, and a peak stress range from about 15 MPa to about 50 MPa, at a strain-at-break of from about 200% to about 1000% of original dimensions. Typically, the modulus is in a range from about 55 MPa or 60 MPa to about 260 MPa or 275 MPa, and more typically from about 67 MPa or 75 MPa to about 225 MPa or 240 MPa, inclusive of any combination of ranges there between. Typically, the peak stress can range from about 20 MPa or 23 MPa to about 40 MPa or 45 MPa, inclusive of any combination of ranges there between.

The polymeric film will tend to have a micro-textured surface with topographic features, such as ridges or bumps, of between about 0.5 micrometers or 1 micrometers up to about 10 micrometers or 12 micrometers in size. Typically the features will have a dimension of about 2 micrometers or 3 micrometers to about 7 micrometers or 8 micrometers, or on average about 4 micrometers, 5 micrometers, or 6 micrometers. The particular size of the topographic features will tend to depend on the size of the individual thermoplastic starch particles, and/or their agglomerations and also the process conditions used to fabricate the overall film(s).

In contrast to others, which describe rigid injection molding products, the present invention can be used to create thin flexible films based on polyolefins and TPS masterbatch, which are more suited to the specific requirements of packaging films.

In another aspect, the invention describes a method of forming a polymeric film. The method comprising: preparing a polyolefin mixture, blending said polyolefin mixture with a thermoplastic starch and a compatibilizer, which is a graft copolymer of a non-polar backbone and a grafted polar monomer or a block copolymer of both a non-polar block and a polar block or a random copolymer of the non-polar monomer and a polar monomer, said thermoplastic starch and compatibilizer, respectively, are present in amounts in a ratio of between about 2.5:1, 5:1, 7.5:1 10:1, 15:1, 30:1 or about 95:1; extruding said a film of said blended polymer mixture. Desirably, the compatibilizer includes a graft copolymer of polyethylene and maleic anhydride, polyethylene-co-acrylic acid (EAA), polyethylene-co-vinyl alcohol (EVOH), polyethylene-co-vinyl acetate (EVA).

Alternatively, the method of forming a polymeric film may include the steps of preparing a polyolefin mixture; blending the polyolefin mixture with a TPS masterbatch or concentrate; and extruding said mixture to form a film of said blended polymer mixture. The TPS masterbatch or concentrate and polyolefins, respectively, are present in amounts in a ratio of between about 1:1 to about 0.1:1.

In contrast to other methods of preparing thermoplastic starch and synthetic polymer blends, no water-based suspension, evaporation step is needed in the present invention. Also, the present invention does not employ starch-polyester graft copolymers.

The following description and examples will further illustrate the present invention. It is understood that these specific embodiments are representative of the general inventive concept.

### A. Blends of Polyolefin and Thermoplastic Starch

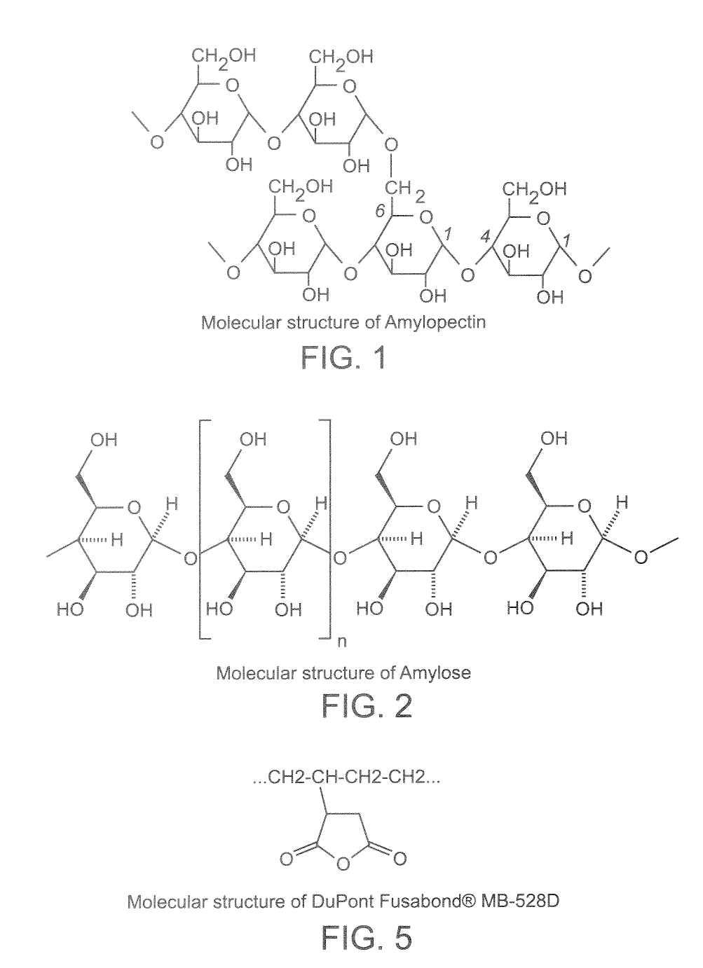

For purposes of illustration, TPS samples are prepared with a twin-screw compounding extruder. As an example, cornstarch is incorporated at about 50 or 70 wt. % to about 85 or 90 wt. %, and a plasticizer, such as glycerol or sorbitol, is added up to about 30 or 33 wt. %. A surfactant, such as Excel P-40S, is added to help lubricate the thermoplastic mixture. The mixture is extruded under heat and mechanical shear to form TPS. Blending the TPS with a polyolefin (e.g. LLDPE, LDPE, HDPE, PP, etc.) polymer produces films with un-dispersed aggregates of TPS in the films. The thermoplastic starch and polyolefin are observed to be incompatible with each other. An explanation appears to be found in the molecular structure of each material. The starch is comprised of two components: Amylopectin, which exists as about 70-80% of corn starch's composition, is a highly branched component of starch. Its structure is illustrated in FIG. 1. The remaining percentage (20-30%) of starch's composition is amylose, which is the mostly linear component of starch. Its structure is illustrated in FIG. 2. Both amylopectin and amylose are comprised of glucosidic repeating units that are connected by oxygen atoms (i.e. ether linkages) and they contain a large number of hydroxyl groups. The ratio of amylose to amylopectin comprising a starch varies depending on the type of plant from which it was derived.

In contrast, the molecular structure of polyolefin is a simple saturated hydrocarbon polymer. Polyolefin does not contain any polar functional groups such as hydroxyl groups nor are they linked by oxygen atoms. Thus, mixing of the polyolefin and the thermoplastic starch is not fully homogenous because polyolefin does not contain any polar functional groups that are needed to disperse the thermoplastic starch moieties evenly throughout the film material. Films created from thermoplastic starch and polyolefin alone exhibit many undispersed thermoplastic starch aggregates and holes due to their incompatibility.

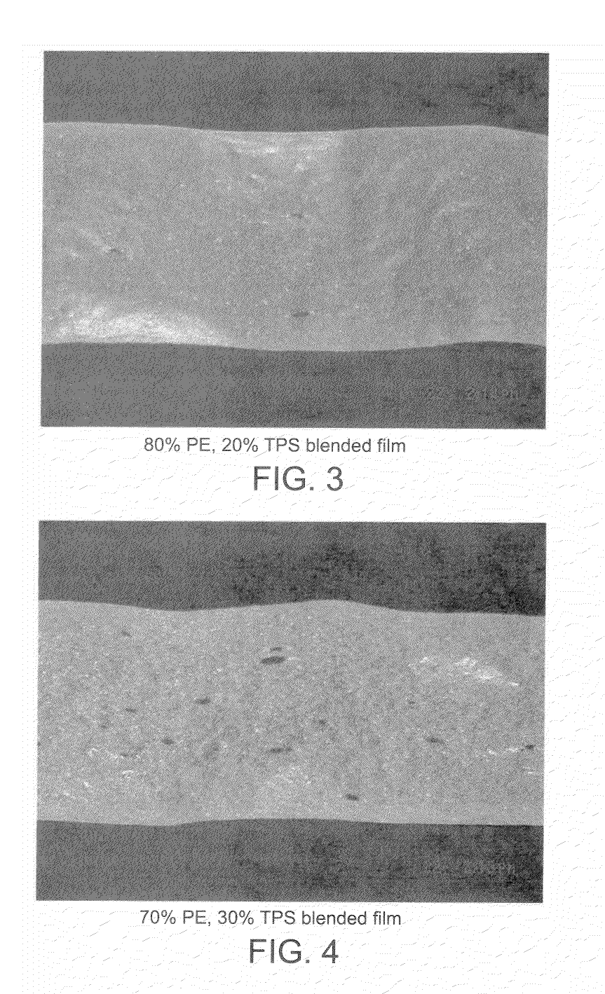

For example, FIG. 3 shows a film blended of 80% by weight (wt.) of PE and 20% by weight of TPS. A number of undispersed TPS (white dots) and holes have developed due to the orientation in the machine direction by the chill roll during film casting. The polyethylene will stretch, but when a chunk of undispersed TPS is encountered, the TPS will not stretch, and will tear a hole in the film membrane. Similar to the film shown in FIG. 3, FIG. 4 shows a film containing 30% (wt.) TPS blended with 70% (wt.) PE. The undispersed TPS aggregates and the large number of holes in the film can be readily observed. The greater the amount of TPS that is added into the film, the worse the film becomes and the more important TPS dispersion becomes.

### B. Compatibilizers

To improve the compatibility and dispersion characteristics of thermoplastic starch in polyolefins, several compatibilizers with both polar and non-polar groups are incorporated in the present invention. The compatibilizers may include several different kinds of copolymers including graft copolymers having a non-polar backbone and a grafted polar monomer or a block copolymer of a non-polar block and a polar block, or a random copolymer of a non-polar monomer and a polar monomer, for example, polyethylene-co-vinyl acetate (EVA), polyethylene-co-vinyl alcohol (EVOH), polyethylene-co-acrylic (EAA), and a graft copolymer of a polyolefin (e.g., polyethylene or polypropylene) (e.g., DuPont Fusabond® MB-528D) and maleic anhydride based on molecular structure considerations. EVA, EVOH, EAA, etc. both have a non-polar polyethylene subunit in their backbones. The vinyl acetate subunit contains an ester group, which can hydrogen bond with the hydroxyls of the amylopectin and amylose. EVOH has a vinyl alcohol group, which can hydrogen bond with the hydroxyl groups in starch. The ester group in EVA and the hydroxyl group in EVOH do not chemically react with the hydroxyl groups in starch molecules. Instead, they associate with starch through hydrogen bonding or polar-polar molecular interactions. Using these two physical compatibilizers, blends of TPS and EVA or TPS and EVOH, showed improved compatibility versus the un-compatibilized PE/TPS blends.

As a graft copolymer of polyethylene and maleic anhydride, Fusabond® MB-528D has a structure shown in FIG. 5. The cyclic anhydride at one end is chemically bonded directly with the polyethylene chain. The polar anhydride group of the graft copolymer molecule could associate with the hydroxyl groups in the starch via both hydrogen bonding and polar-polar molecular interactions and/or a chemical reaction to form an ester linkage during the melt extrusion process. The hydroxyls of the starch will undergo esterification reaction with the anhydride to achieve a ring-opening reaction to chemically link the thermoplastic starch to the maleic anhydride to the grafted polyethylene. This reaction is accomplished under the high temperatures and pressures of the extrusion process.

The EVA and EVOH worked sufficiently well to disperse the starch particles. In comparison to the graft copolymer of polyethylene and maleic anhydride, however, EVA and EVOH, even at higher percentages of about 10 or about 15%, did not fully disperse the TPS in the film. DuPont Fusabond® MB-528D, however, completely dispersed the TPS in the film when present at a concentration of about 1% to about 5%. Hence, the graft copolymer of polyethylene and maleic anhydride appears to be a more effective compatibilizer.

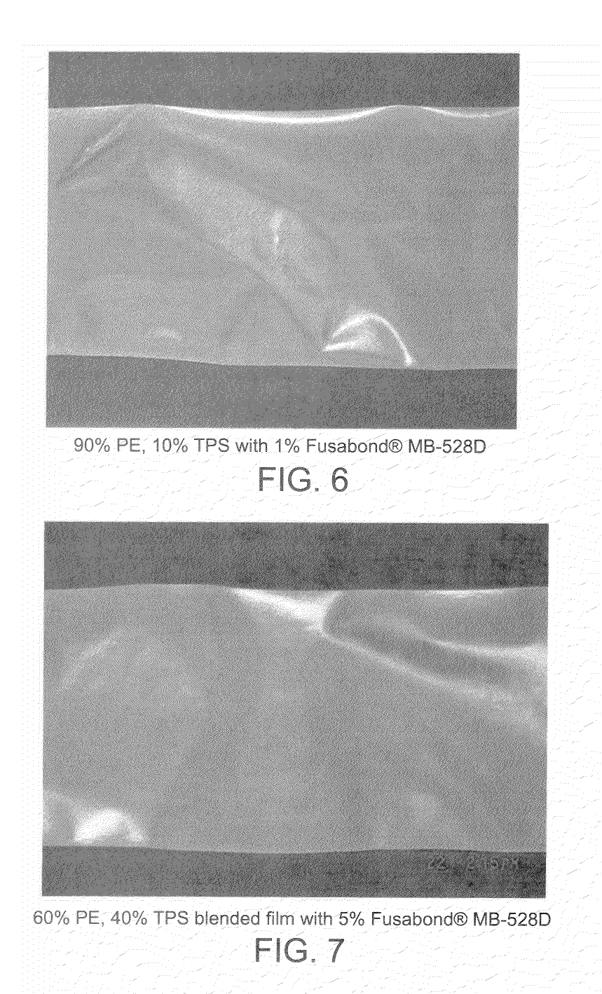

An example of a film made according to the present invention, shown in FIG. 6, contains about 90% PE and 10% TPS blended with 1% Fusabond® MB-528D, a compatibilizer. The compatibilizer helps the TPS fully disperse into the polyolefin. The undispersed TPS that was previously seen in the films is nonexistent, since the starch has been fully dispersed into the polyethylene. Another example is the film shown in FIG. 7, which contains about 60% PE and 40% TPS blended with 5% Fusabond® MB-528D. Similar to FIG. 6, the film showed no undispersed starch aggregates and no holes. The thermoplastic starch was fully homogenized up to 40%.

The graft copolymer of polyethylene and maleic anhydride appears to better compatibilize blends when a melt blended resin was made on a ZSK-30 twin screw extruder. In comparison, dry blends with the compatibilizer did not give the same homogenization as the extrusion melt compounded resin. The dry blends are placed directly into the hopper of a HAAKE single screw extruder, but the machine did not exhibit the same shear provided by the twin screws on the ZSK-30 extruder. The twin screw, along with specific mixing capability of the screws, provides a much more effective mixing of all the ingredients. This same mixing cannot be accomplished on the HAAKE extruder.

### C. Dispersion

When the graft copolymer of polyethylene and maleic anhydride, Fusabond® MB-528D, disperses the TPS, it does so partially by chemical reaction. Therefore, a stoichiometric amount of Fusabond® MB-528D will provide ample homogenization to the film. Generally, the more TPS content that is added in the blend, the more Fusabond® MB-528D needs to be added to provide sufficient bonding sites for the hydroxyl groups of the starch molecule. When different Fusabond® MB-528D ratios are tried, two types of undispersed polymer aggregates tend to form: TPS aggregates, which are yellowish accumulations of TPS in the film, and Fusabond® MB-528D aggregates. The second aggregate type forms when too much Fusabond® MB-528D is added to the film; the Fusabond® will not be fully dispersed. A control was prepared to show this effect. LLDPE was mixed with Fusabond® MB-528D at 2.5%. The film produced showed clear polymer aggregates and streaks, which is a sign of unreacted Fusabond®. For each particular ratio of PE to TPS, there is a specific amount of the Fusabond® compatibilizer that will provide successful dispersion for all components of the film.

According to the present invention, the amount of polyolefin and compatibilizer, present in the composition can be expressed as a ratio of between about 5:1 or about 6:1 to about 90:1 or about 95:1, or any combination or permutation of ratio values there between. Alternatively, the ratio may be, for instance, between about 10:1 or about 12:1 to about 60:1 or about 70:1, or preferably between about 15:1 or about 17:1 to about 50:1 or about 55:1, or more preferably between about 20:1 or about 22:1 to about 40:1 or about 45:1 (e.g., 25:1, 27:1, 30:1, 33:1, or 35:1).

FIG. 8 is a graph that shows the dispersion region for relative incorporated amounts of a compatibilizer (i.e. Fusabond®) as a function of the polyolefin content in several different blends. The upper and lower solid lines represent the respective upper and lower limits of compatibilizer solubility. The region between the upper and the lower solid lines represents the acceptable zone in which the compatibilizer can be incorporated with best results. In other words, if the amount of compatibilizer added is greater than that of the upper limit line, the compatibilizer will not disperse evenly throughout the blend composition. If the compatibilizer content is less than that of the lower limit line, then regions of undispersed thermoplastic starch particles will tend to aggregate in the film.

### D. Physical Properties of Polymeric Film

The polymeric films are subjected to tensile testing to evaluate their physical properties. FIG. 9, shows the moduli of five films with different levels of TPS incorporation. There are two sets of data on these graphs because there are two directions to test on the film. MD is the machine direction, and that is the direction that is parallel with the film movement exiting the extruder. CD is the cross direction which is perpendicular to the direction of film movement. In both directions (MD and CD), the film became more rigid as more TPS was incorporated. TPS is inherently very brittle and its molecular structure determines its low flexibility. Therefore, the more TPS in a blend, the more rigid it is expected to be. When up to 40% TPS was added, the modulus in both directions more than doubled that of the LLDPE control. Also, there was little difference between the control and the 90/10 (all the ratios are weight ratios) PE/TPS blend data. This showed that when a small amount of TPS are added to the film, it had little effect. However, once up to about 20% TPS was added, there was a large jump in the modulus. Even with this modulus increase, the films were still relatively soft.

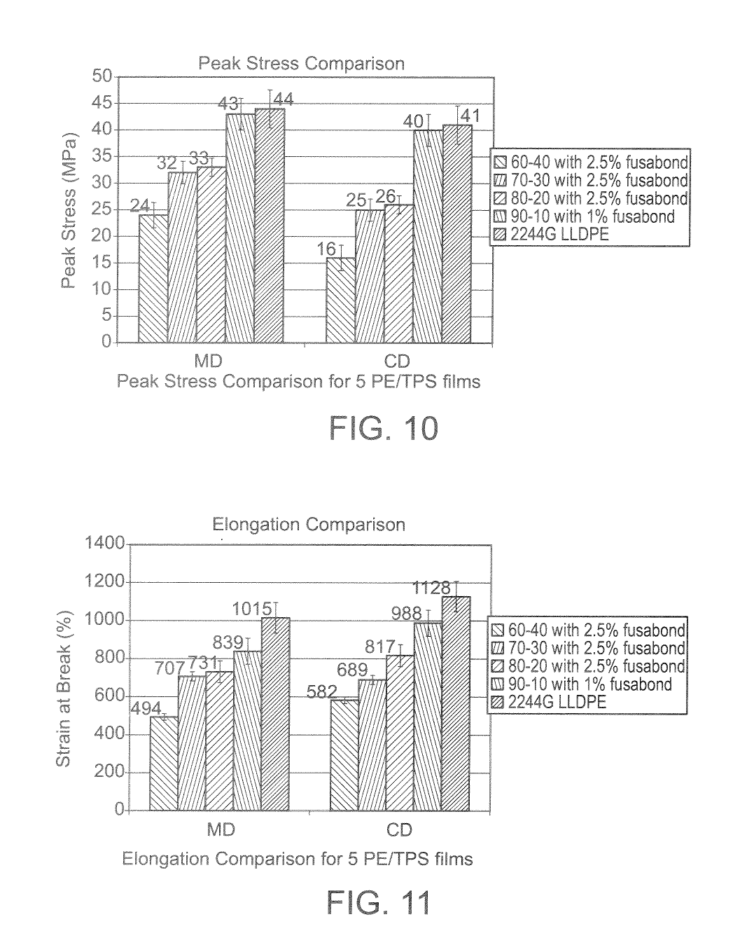

FIG. 10, shows the peak stress of the same five films as in FIG. 9. Again, the 90/10 blend is very close to the control. As more TPS was added into the film, the film became weaker. This is due to the fact that TPS, again, does not make a very strong, flexible plastic film. The 60/40 blend in both directions was approximately half as strong as the LLDPE film control.

FIG. 11 shows the strain-at-break of these five film samples from FIGS. 9 and 10. As more TPS was added to the LLDPE, the film's strain-at-break decreases. The strain-at-break for the 90/10 blend was not as close to the control as the previous modulus and peak stress data has shown. Its strain-at-break however was still very high. There was a general constant difference between each blend as 10% more TPS was added. At 30 and 40% TPS, the strain-at-break was around two-thirds to one-half the strain-at-break of the LLDPE control. The physical data of these two blends was substantially low when compared to the LLDPE control film. The observed strain-at-break of 500-700%, although much lower than the LLDPE control film data, were still significantly high to be useful for many packaging film applications.

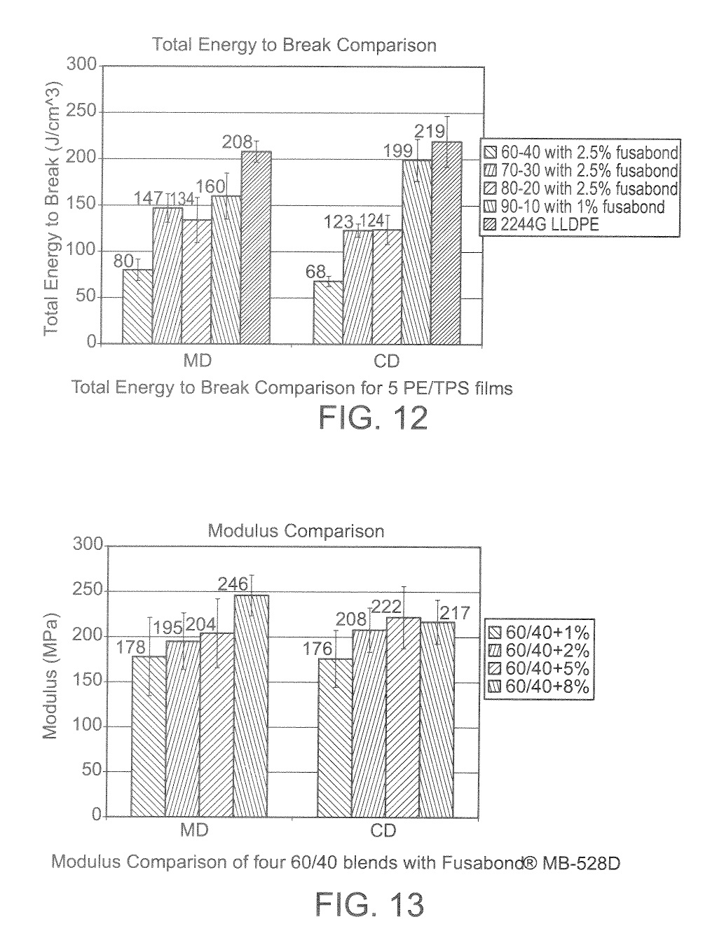

FIG. 12 shows the energy-to-break the partially renewable films by stretching along machine direction (MD) and cross-direction (CD). Significantly less energy was needed to break the blend films comprising 40% TPS versus the films comprising 20%-30% TPS.

### E. Effect of Compatibilizer on Physical Properties of Films

Adding Fusabond® MB-528D as a compatibilizer has effects on the physical properties of the film. It chemically bonds the grafted LLDPE to the TPS. The more bonds that are formed in the film, the more rigid the film will become. The effects of this compatibilizer can be seen from the following tensile data.

FIG. 13 shows the moduli of four 60% PE, 40% TPS films that were blended with different percentage amounts of compatibilizer (Fusabond® MB-528D). Each ratio is shown in the legend. As more compatibilizer was added, the more rigid the film became due to increased level of reaction. The green bar with 1% Fusabond® MB-528D is much softer than the middle two blends. This ratio, however, was not in the window of dispersion, and therefore it is not a recommended blend. The 8% compatibilizer blend did not possess any undispersed polymer at 60/40 PE/TPS ratio.

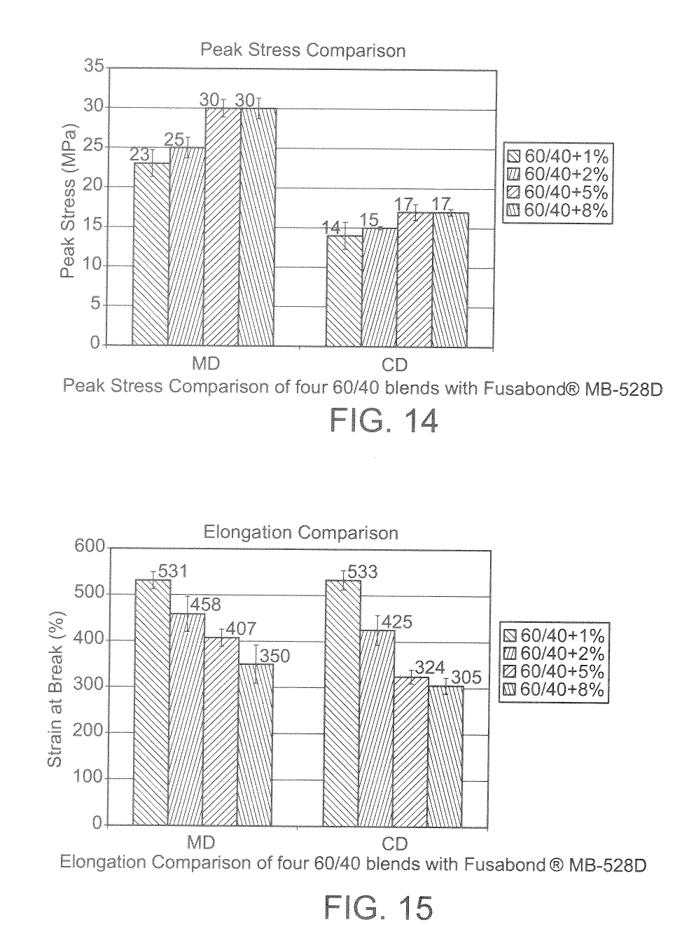

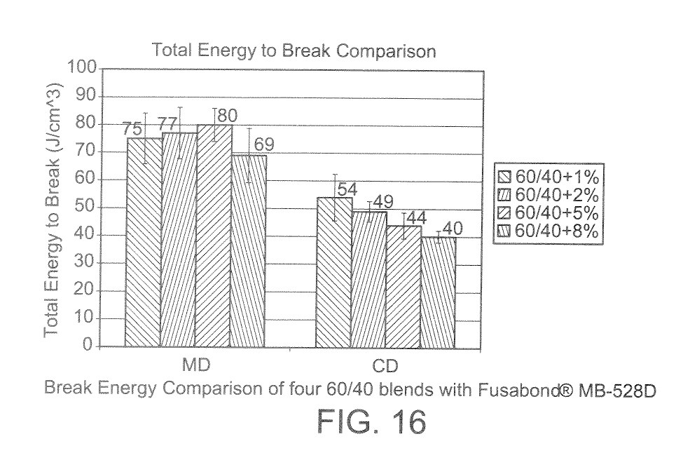

FIG. 14 is a graph that shows the peak stresses of these same four blends. Similar in trend, the strength of the film was increased as more Fusabond® MB-528D was added to the film. FIG. 15 is a graph that summarizes the strain-at-break of the four blend films of FIG. 13. As the films become more rigid, they do not stretch as far. There was a significant difference in the film properties when the amount of Fusabond® MB-528D is at 1 wt. % versus at 8 wt. %. The 60/40 blend at 1 wt. % did not disperse all the starch throughout the film, so the undispersed thermoplastic starch did not become part of the film. Undispersed aggregates have a tendency to weaken the film when stretched. At higher concentrations (e.g., ≧5 wt. %), the film is observably more flexible and pliant. The graph shows that the lower the amount of compatibilizer and TPS that is mixed with the PE, the more it becomes like the control sample, which is pure PE, since proportionately, the PE phase is a more dominant component in the polymer matrix than the compatibilizer in terms of contribution to the films' properties. Nonetheless, even with a small amount (e.g., ˜1-2%) mixed in the blend, as shown, the film exhibited a more flexible and uniform appearance than without the compatibilizer. FIG. 16 is a graph that shows the break energy of these films. In the cross direction, less energy was required to break the film as the amount of the compatibilizer is increased.

### F. Illustrative Consumer Product

The present thermoplastic film materials can be used to make packaging for various kinds of consumer products in general terms. For purpose of illustration, certain package embodiments may be for health care products or consumer products such as absorbent articles (e.g., baby diapers or feminine hygiene articles). The package can have one or more absorbent articles disposed therein. As used herein, the term “absorbent article” refers to devices that absorb and/or contain a substance such as body exudates. A typical absorbent article can be placed against or in proximity to the body of the wearer to absorb and contain various body excretions such as in diapers, incontinence articles, feminine hygiene articles and the like.

### G. Materials

### Dowlex 2244G Polyethylene Resin

Linear low density polyethylene produced by The Dow Chemical Company, Midland, Mich. This resin was used as the main, nonrenewable component of the partially renewable films.

### Cornstarch

Produced by Cargill, Inc. Hammond, Ind. This was the native cornstarch source used to produce the homemade TPS.

### D-Sorbitol

Plasticizer purchased from Sigma-Aldrich, St. Louis, Mo. Sorbitol was used at 30% along with cornstarch while compounding the thermoplastic starch.

### Excel P-40S

Surfactant produced by The Kao Corporation, Tokyo, Japan. Surfactant was added at 2% to lubricate the polymer and reduce torque on the extruder screws.

### DuPont Fusabond® MB-528D

Compatibilizer produced by DuPont Canada Company, Mississauga, Ontario. Fusabond® MB-528D is >99% maleic anhydride modified polyethylene (LLDPE). Used as a compatibilizer.

### Escorene® Ultra Ethylene Vinyl Acetate

Produced by ExxonMobil Chemical Company, Houston, Tex. EVA was tried as a potential compatibilizer. It contained<0.2% vinyl acetate.

### Ethylene Vinyl Alcohol Copolymer

Produced by EVAL Company of America, Houston, Tex. This is a copolymer of ethylene and vinyl alcohol via EVA.

### Compounding

Blended resins are made on the ZSK-30 Twin Screw Extruder. TPS was prepared according to U.S. patent application Ser. No. 11/640,109 by Wang et al. TPS was fed by one feeder and a blend of 2244G LLDPE and Fusabond® MB-528D were fed by another. The dry blend of LLDPE and Fusabond® MB-528D was prepared by the addition of compatibilizer such that when fully mixed with TPS, the desired ratio was obtained.

The TPS was often fed by Feeder 2 and the LLDPE/Fusabond® blend was fed by Feeder 3. The ZSK-30 ran at 20 lbs/hr. For 90/10 blends, Feeder 2 was set to 2 lbs/hr and Feeder 3 was set to 18 lbs/hr. The ratios of mass flow rates were adjusted to give the desired ratio of LLDPE and TPS while keeping the overall flow rate of 20 lbs/hr. The temperature profile on the ZSK-30 extruder is shown in Table 1.

TABLE 1
Temperature profile on ZSK-30 for blends

| Zone | Temp (° C.) |
|---|---|
| 1 | 100 |
| 2 | 130 |
| 3 | 175 |
| 4 | 175 |
| 5 | 175 |
| 6 | 175 |
| 7 | 175 |

The melt temperature of the blend, Tₘ=197° C., was approximately the same for all blends. The pressure ranged from 350-500 psi and torque from 60-80%. The compounding screw and a 3-hole die were used for every trial. The screw speed was set to 200 rpm. The resin strands produced by the ZSK-30 were cooled on a cooling belt by a series of fans. Once the resin had cooled, it was pelletized and placed in a bag for shipping.

The processing conditions for TPS alone are different than that for the LLDPE/TPS blending. The temperature profile on the ZSK-30 extruder is shown in Table 2.

TABLE 2
Temperature profile on ZSK-30 for TPS

| Zone | Temp (° C.) |
|---|---|
| 1 | 95 |
| 2 | 110 |
| 3 | 115 |
| 4 | 120 |
| 5 | 120 |
| 6 | 120 |
| 7 | 115 |

The screw speed was set to 150 rpm and the pressure ranged from 700-1300 psi. The melt temperature, Tm was 130° C. and the torque ranged from 30-47%. The powder feeder was used and ran at 20 lbs/hr. A nip was used to draw down the stands of the TPS before being pelletized.

### Film Casting

All films were cast on the HAAKE Rheomex 252 Single Screw Extruder. A chill roll was used to cool the polymer as it came from the cast film die and to flatten it to form the thin film. The processing conditions for the extruder were the same for all films cast. They were as follows is shown in Table 3.

TABLE 3
Temperature profile on HAAKE for film casting

| Zone | Temp (° C.) |
|---|---|
| 1 | 150 |
| 2 | 160 |
| 3 | 170 |
| 4 | 170 |
| 5 | 150 |

The screw speed was set to 50-60 rpm. The pressure was kept around 1000 psi and the torque ranged between 3000-4000 m·g. The chill roll settings were adjusted as needed to obtain films with a gauge of 2.0 mil. If the film was too thick, the chill roll was sped up to draw the polymer out of the die faster, making a thinner film. If the film was too thin, the chill roll was slowed down.

The HAAKE extruder has fewer temperature zones than the ZSK-30 extruder. This is because the ZSK-30 has much longer screws than the HAAKE, so more zones are needed to obtain the same accuracy of the temperature distribution.

### Dispersion Window

Each data point on the graph in FIG. 8, represents a film that was cast in the lab. If the film had no undispersed polymer, that ratio was placed in the window of dispersion. If clear polymer aggregates were seen, that blend was placed outside the window. Similarly, if yellow aggregates were seen, that means the starch was not fully dispersed, and the blend was placed outside the window. Approximately four blend ratios were tried for each PE amount (60%, 70%, 80%, and 90%). The control, LLDPE, did not contain any other components, and thus did not require a compatibilizer. Based on the quality and evaluation of the films, it can be determined by the data points of FIG. 8, the optimal inclusion of all three elements (polyolefin, TPS masterbatch, and compatibilizer) of the film. (FIG. 8). Above the optimal range of the compositions, there is the presence of undispersed compatibilizer. Below the optimal range is shown undispersed TPS gels. The middle of FIG. 8 shows the optimal ranges and percentage that should be included in the present invention in order to ensure that the desired attributes of the invention are met. In other words, the three components cannot simply be blended together to arrive at the present invention. Rather, there must be the exact ratios and ranges that are presently disclosed in order to achieve the desired results, namely good mechanical properties, good processability, and to be free from any visible defects. FIG. 8, therefore, also shows the ratio in which the undispersed polymer became visible. The upper limit for the 60/40 blend was not reached during the experiments. No polymer blend containing more than 8%, by weight of Fusabond® MB-528D, was prepared. Even at a high level, no undispersed Fusabond® MB-528D was observed, which may be due to the high amount of TPS present in the blend. The starch hydroxyls were still able to provide reaction sites with the maleic anhydride.

### Tensile Property Test

All tensile properties were tested on the MTS Sintech 1/D tensile testing apparatus. Samples were prepared for testing by taking a portion of the film, and cutting five dog-bone shaped samples in each direction (i.e., machine direction (MD) and cross-machine direction (CD)). The test length of each dog-bone was 18 mm, the width of the test area was 3 mm, and the thickness varied by about 2 mil. Each dog-bone was tested separately. During the test, samples were stretched at a crosshead speed of 5.0 inches/minute until breakage occurred. The computer program TestWorks 4 collected data points during the testing and generated a stress (MPa) versus strain (%) curve from which a variety of properties were determined: modulus, peak stress, strength-at-break, and energy-to-break.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Comparative Example

A mixture of 60% of a thermoplastic starch masterbatch (BL-F, produced by Cardia, formerly Biograde, Nanjing, China), 32% of a linear low density polyethylene (LLDPE) (melt flow rate of 1 and density of 0.918 g/cc, Grade 118 W, supplied by SABIC), and 8% white master batch (Shanghai Ngai Hing Plastic Materials Co., Ltd.) was fed to a three-layer blown film line. The extruders had a screw diameter of 250 mm, and a Length/Diameter of 30/1. The die gap was 2.2 mm.

The film extrusion conditions are listed in the following table:

| Temperature | Screw | Screw | Screw | Screw | |
|---|---|---|---|---|---|
| (° C.) | Section I | Section II | Section III | Section IV | Die |
| Outer-layer | 155 | 165 | 165 | 164 | 165 |
| Middle-layer | 155 | 165 | 165 | 165 | 160 |
| Inner-tier | 155 | 165 | 165 | 165 | 160 |

Unlike conventional polyethylene-based films, biodegradable polymeric films according to the present invention exhibit a more micro-textured surface.

1. Tensile test results:

| | Tensile | Tensile | % Elongation | % Elongation |
|---|---|---|---|---|
| | Strength MD | Strength CD | at Break Point | at Break Point |
| | (N/15 mm) | (N/15 mm) | MD | CD |
| Tensile | 12 | 14 | 213 | 16 |
| Test | | | | |

The tensile properties of the comparative films were very poor for packaging film applications. The film ripped easily.

### Example 1

A mixture of 17% of a TPS masterbatch (BL-F, produced by Cardia, formerly Biograde, Nanjing, China), 38% of a linear low density polyethylene (LLDPE) (melt flow rate of 1 and density of 0.918 g/cc, Grade 118 W, supplied by SABIC) and 38% low density polyethylene (LDPE) (melt flow rate of 2.8 g/10 min and density: 0.925, Grade: Q281, supplied by SINOPEC Shanghai, Shanghai, China), and 7% white color masterbatch (Shanghai Ngai Hing Plastic Materials Co., Ltd.) was fed to a single screw extruder blown film machine, the screw diameter was 150 mm, the Length/Diameter was 30/1. The die gap was 1.8 mm.

The other process conditions are listed in the following table:

| | Temperature | | | | | | | |
|---|---|---|---|---|---|---|---|---|
| | NO. 8 | NO. 7 | NO. 6 | NO. 5 | NO. 4 | NO. 3 | NO. 2 | Die |
| | HEATER | HEATER | HEATER | HEATER | HEATER | HEATER | HEATER | Temperature |
| | (° C.) | (° C.) | (° C.) | (° C.) | (° C.) | (° C.) | (° C.) | (° C.) |
| Example 1 | 180 | 180 | 180 | 173 | 164 | 160 | 147 | 184 |
| Example 2 | 180 | 180 | 180 | 173 | 164 | 160 | 147 | 180 |
| Example 3 | 180 | 180 | 180 | 173 | 164 | 160 | 147 | 180 |

### Example 2

A mixture of 37% of a TPS masterbatch (BL-F, produced by Biograde, Nanjing, China), 28% of a linear low density polyethylene (LLDPE) (melt flow rate of 1 and density of 0.918 g/cc, Grade 118 W, supplied by SABIC) and 28% low density polyethylene (LDPE) (melt flow rate of 2.8 g/10 min and density: 0.925, Grade Q281, supplied by SINOPEC Shanghai, Shanghai, China), and 7% white masterbatch (Shanghai Ngai Hing Plastic Materials Co., Ltd.) was fed to a single screw extruder blown film machine, the screw diameter was 150 mm, the Length/Diameter was 30/1. The die gap was 1.8 mm.

### Example 3

A mixture of 57% of a TPS masterbatch (BL-F, produced by Cardia, formerly Biograde, Nanjing, China), 18% of a linear low density polyethylene (LLDPE) (melt flow rate of 1 and density of 0.918 g/cc, Grade 118 W, supplied by SABIC) and 18% low density polyethylene (LDPE) (melt flow rate of 2.8 g/10 min and density: 0.925, Grade: Q281, supplied by SINOPEC Shanghai, Shanghai, China), and 7% white masterbatch (Shanghai Ngai Hing Plastic Materials Co., Ltd.) was fed to a single screw extruder blown film machine, the screw diameter was 150 mm, the Length/Diameter was 30/1. The die gap was 1.8 mm.

All the films from Examples 1, 2, and 3 were printed with conventional dyes/inks used in packaging. The printing quality of Example 1 appeared to be the best. These films were also converted into product bags for absorbent products, and no physical or visual issues were encountered. The winding tension was reduced from 10.6 kgf to 6.1 kgf to overcome wrinkle issues. Mechanical and other physical testing were performed, the results were listed in the following tables:

| | Tensile | Tensile | % Elongation | % Elongation |
|---|---|---|---|---|
| | Strength MD | Strength CD | at Break Point | at Break Point |
| | (N/25.4 mm) | (N/25.4 mm) | MD | CD |
| Example 1 | 28.7 | 26.5 | 687 | 735 |
| Example 2 | 24.1 | 20.4 | 591 | 624 |
| Example 3 | 18.4 | 15.5 | 316 | 214 |

### Printed Dots Loss in a Printing Test:

The printed film in Example 2 after being subjected to an ink loss test, the results are listed in the following table:

| | Original Dot Design | | | | | | |
|---|---|---|---|---|---|---|---|
| | 100% | 90% | 80% | 75% | 70% | 60% | 50% |
| Loss % | 0 | 0 | 5 | 7 | 10 | 15 | 20 |
| | Original Dot Design | | | | | | |
| | 40% | 30% | 25% | 20% | 15% | 10% | 5% |
| Loss % | 30 | 50 | 60 | 70 | 80 | 90 | 100 |

### Rapid Aging Test (RAT):

Test condition

| | | Testing | | |
|---|---|---|---|---|
| | Test condition | Equipment | Tested samples | Test Period |
| RAT I | 54-47° C. | oven | Example 1 | 14 days |
| | 54-47° C. | oven | Example 2 | 14 days |
| RAT II | 37-40° C. | oven | Example 1 | 3 months |
| | 37-40° C. | oven | Example 2 | 3 months |
| RAT | 54-47° C., >75% | CTCH | Example 1 | 14 days |
| III | Relative | | | |
| | Humidity | | | |
| | 54-47° C., >75% | CTCH | Example 2 | 14 days |
| | RH | | | |
| RAT | 37-40° C., >75% | CTCH | Example 1 | 3 months |
| IV | RH | | | |
| | 37-40° C., >75% | CTCH | Example 2 | 3 months |
| | RH | | | |

Note:
CTCH: Constant temperature and constant humidity.

Mechanical test results:

| | | | | % |
|---|---|---|---|---|
| | Tensile | Tensile | % Elongation | Elongation |
| | Strength MD | Strength CD | at Break Point | at Break |
| Performance | (N/25.4 mm) | (N/25.4 mm) | MD | Point CD |
| RAT I-80% | 28.3 | 26.0 | 695 | 663 |
| RAT I-60% | 20.8 | 19.7 | 348 | 270 |
| RAT II-80% | 27.5 | 24.0 | 675 | 696 |
| RAT II-60% | 21.1 | 18.3 | 451 | 467 |
| RAT III-80% | 24.2 | 29.2 | 692 | 712 |
| RAT III-60% | 22.3 | 22.3 | 338 | 201 |
| RAT IV-80% | 25.0 | 30.5 | 718 | 726 |
| RAT IV-60% | 20.2 | 31.4 | 303 | 424 |

### Submersion Test:

Considering the renewable film package will be stored or used in places with high humidity, such as lavatories or bathrooms, a hot water vapor and/or liquid submersion test was conducted to test how well the films may withstand liquid water or water vapor. Since the films according to the present invention contain TPS that is water sensitive, it was expected that the tensile strength of the films would be easier to compromise when exposed to or immersed in water. The results are summarized in the following tables. A finding of interest is that the MD/CD tensile strength and elogation percentage values are even better that those samples that were not subjected to the water vapor or liquid immersion.

Test Condition

| | | Testing | | Test |
|---|---|---|---|---|
| | Test condition | Equipment | Tested samples | Period |
| Test I | 20° C. water | Container | 55 μm: Example 1 | 24 hours |
| | steam | | 45 μm: Example 1 | |
| Test II | 20° C. 9% salt | Container | 55 μm: Example 1 | 24 hours |
| | aqueous solution | | 45 μm: Example 1 | |

Performance Test Result

| | | | | % |
|---|---|---|---|---|
| | Tensile | Tensile | % Elongation | Elongation |
| | Strength MD | Strength CD | at Break | at Break |
| Performance | (N/25.4 mm) | (N/25.4 mm) | Point MD | Point CD |
| Test I-55 μm | 31.2 | 31.3 | 652 | 648 |
| Test I-45 μm | 25.3 | 25.8 | 590 | 580 |
| Test II-55 μm | 25.8 | 24.8 | 719 | 689 |
| Test II-45 μm | 20.9 | 20.2 | 650 | 639 |

### Example 4

This example demonstrates a three layer film made on a pilot blown film extrusion line. In this example, the exterior layers A and C are identical and comprise of 45% Dow LLDPE 2085B (density 0.919), 45% Dow LMDPE 2038.68G (density: 0.935), and 10% Dow LDPE 501I. The interior layer contains 32% Dow 2085B, 32% 2038.68G, 10% Dow 501I, and 26% of Biograde BL-F resin. The film had an overall 10% by weight of plant starch-based material.

The co-extrusion was process on a three-layer blown film line, the extruders for Layer A and Layer C were single screw extruders manufactured by Collins which had a diameter of ¾″ and L/D of 26:1 D. The core layer (Layer B) extruder was a single screw extruder with a diameter of 1.5″ and an L/D of 28/1, manufactured by Killion. The processing temperatures for Layer A (heat heal layer) were: 70, 175, 205, 230, 212, 212, and 213° C. respectively for zones 1 to 6 and the melt temperature, the melt pressure was 167 bar. The processing temperatures for Layer B were: 245, 280, 320, 340, 340, 340, and 319° F. respectively for zones 1 to 3, Die 1 to 3, and melt temperature, the melt pressure was 2900 psi. The processing temperatures for Layer C (the printing surface) were: 95, 175, 205, 230, 212, 212, and 217° C. respectively for zones 1 to 6 and the melt temperature, the melt pressure was 84 bar. The die was capable of producing films of 20/60/20 configuration, the upper block temperature, lower block temperature, adaptor, clamp ring temperatures were 335° C., 335° C., 335° C., and 340° C.

### Example 5

This example demonstrates a three layer film made on a pilot blown film extrusion line. In this example, the exterior layers A and C are identical and comprise of 45% Dow LLDPE 2085B (density 0.919), 45% Dow LMDPE 2038.68G (density: 0.935), and 10% Dow LDPE 501I. The interior layer contains 32% Dow 2085B, 32% 2038.68G, 10% Dow 501I, and 26% of Biograde BL-F resin. The 3-layer film had 20% by weight of plant starch based materials based on the total weight of the films.

The co-extrusion was process on a three-layer blown film line, the extruders for Layer A and Layer C were single screw extruders manufactured by Collins which had a diameter of ¾″ and L/D of 26:1 D. The core layer (Layer B) extruder was a single screw extruder with a diameter of 1.5″ and an L/D of 28/1, manufactured by Killion. The processing temperatures for Layer A (heat heal layer) were: 70, 175, 205, 230, 212, 212, and 213° C. respectively for zones 1 to 6 and the melt temperature, the melt pressure was 167 bar. The processing temperatures for Layer B were: 245, 280, 320, 340, 340, 340, and 319° F. respectively for zones 1 to 3, Die 1 to 3 and melt temperature, the melt pressure was 2900 psi. The processing temperatures for Layer C (the printing surface) were: 95, 175, 205, 230, 212, 212, and 217° C. respectively for zones 1 to 6 and the melt temperature, the melt pressure was 84 bar. The die was capable of producing films of 20/60/20 configuration, the upper block temperature, lower block temperature, adaptor, clamp ring temperatures were 335° C., 335° C., 335° C., and 345° C.

As one incorporates more corn resin into the blend the films become more bio-degradable. Even though embodiments of the present film materials that have a heightened level of starch within will tend to have rougher film surfaces (on a micron scale) than other polyolefin-based packaging film materials, any difference in appearance of finely printed designs or pattern details are virtually imperceptible to the naked eye. Mechanical performance of the film is within commercial tolerances. Favored features of certain film embodiments (e.g., Example 1) have a natural matte finish and a “soft” feel to the touch that is preferred by consumers.

The present invention has been described in general and in detail by way of examples. Persons of skill in the art understand that the invention is not limited necessarily to the embodiments specifically disclosed, but that modifications and variations may be made without departing from the scope of the invention as defined by the following claims or their equivalents, including other equivalent components presently known, or to be developed, which may be used within the scope of the present invention. Therefore, unless changes otherwise depart from the scope of the invention, the changes should be construed as being included herein.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

We claim:

1. A multiple layer polymeric film comprising at least three layers wherein at least two layers comprise at least one polyolefin and a third layer comprises from about 5% to about 45% of a thermoplastic starch, from about 55% to about 95% of at least one polyolefin, and from about 0.5% to about 10% of a compatibilizer, wherein said compatibilizer is selected from the group consisting of a graft copolymer, a block copolymer, and a random copolymer of non-polar monomers and polar monomers.

2. The multiple layer polymeric film of claim 1, wherein said compatibilizer is selected from a graft copolymer of a non-polar backbone and a grafted polar monomer, a block copolymer of a non-polar block and a polar block, and a random copolymer of a polar monomer and a non-polar monomer.

3. The multiple layer polymeric film of claim 1, wherein said thermoplastic starch and compatibilizers, respectively, are present in a ratio of from about 2.5:1 to about 95:1

4. The multiple layer polymeric film of claim 1, wherein said thermoplastic starch and compatibilizers, respectively, are present in a ratio of from about 10:1 to about 30:1.

5. The multiple layer polymeric film of claim 1, wherein said thermoplastic starch comprises a native starch wherein said native starch is selected from corn, wheat, potato, rice, tapioca, and cassava.

6. The multiple layer polymeric film of claim 1 wherein said thermoplastic starch comprises a modified starch with a plasticizer wherein said modified starch is selected from a starch ester, starch ether, oxidized starch, hydrolyzed starch, crosslinked starch, hydroxyalkylated starch, and carboxymethyl starch.

7. The multiple layer polymeric film of claim 1, wherein said thermoplastic starch comprises from about 55 to about 95% starch; from about 5% to about 45% plasticizer; wherein said plasticizer comprises at least one plasticizer selected from polyhydric alcohols including glycerol, glycerine, ethylene glycol, polyethylene glycol, and sorbitol; citric acid, citrate, and aminoethanol; and from about 0.5% to about 5% of surfactant.

8. The multiple layer polymeric film of claim 5, wherein the thermoplastic starch comprises from about 55 to 95% starch, from 5 to 45% plasticizers, and from 0.5% to 5% of surfactant.

9. The multiple layer polymeric film of claim 1, wherein said polyolefin is selected from low density polyethylene, high density polyethylene, linear low density polyethylene, linear medium density polyethylene, linear ultra-low density polyethylene, polypropylene, polyolefin elastomers, ethylene copolymers with vinyl acetate, and methacrylate.

10. The multiple layer polymeric film of claim 1, wherein said compatibilizer is selected from ethylene vinyl acetate copolymer (EVA), ethylene vinyl alcohol copolymer (EVOH), ethylene acrylic acid copolymer (EAA), ethylene methacrylic acid copolymer (EMAA) and a graft copolymer of polyethylene and maleic anhydride.

11. The multiple layer polymeric film of claim 1, wherein said polar functional monomer is selected from maleic anhydride, acrylic acid, vinyl acetate, vinyl alcohol, vinyl amine, acrylamide, glycidyl acrylate, and glycidyl methacrylate, and is present in an amount from about 0.1% to about 40% by weight.

12. The multiple layer polymeric flexible film of claim 1, wherein the said film has a combined thickness from about 0.5 mil to about 5 mil.

13. The multiple layer polymer film of claim 1, wherein said film has a micro-textured surface with topographic features of from about 0.5 microns to about 8 microns.

14. A flexible multiple layer polymeric film comprising from about 5% to about 55% of a thermoplastic starch masterbatch and from about 40% to about 95% of a polyolefin or mixtures of polyolefins.

15. A flexible multiple layer polymeric film comprising: from about 5% to about 45% of a thermoplastic starch masterbatch, from about 40% to about 95% of a polyolefin or mixtures of polyolefins, and from about 1% to about 15% of a color concentrate.

16. The polymeric multiple layer flexible film of claim 14, wherein said thermoplastic starch masterbatch comprises from about 50% to about 90% of starch, about 0.5% to about 25% of a polyolefin or mixtures of polyolefins, and about 0.5% to about 10% of a compatibilizer selected from a graft copolymer of a non-polar backbone and a grafted polar monomer, a block copolymer of a non-polar block and a polar block, a random copolymer of a polar monomer and a non-polar monomer.

17. A packaging assembly for a consumer product, said packaging comprising at least a portion made from the multiple layer polymeric film of claim 1.

18. A consumer product comprising a portion made from the multiple layer polymeric film of claim 1, wherein said consumer product is an absorbent article, wherein said absorbent article is selected from diapers, pantiliners, feminine pads, adult incontinence products.

19. The consumer product of claim 18, wherein said polymeric film comprises from about 5% to about 45% of a thermoplastic starch, from about 55% to about 95% of a polyolefin or mixtures of polyolefins, and from about 0.5% to about 10% of a compatibilizer selected from a graft copolymer of a non-polar backbone, and a grafted polar monomer, a block copolymer of a non-polar block and a polar block, a random copolymer of a polar monomer and a non-polar monomer, said thermoplastic starch and compatibilizer, respectively, being present in a ratio of from about 2.5:1 to about 95:1.

20. The multiple layer polymeric film of claim 1 wherein the film layers have a thickness of from about 0.05 mil to about 2 mil.

21. The multiple layer polymeric film of claim 14 wherein the film layers have a combined thickness of from about 0.5 mil to about 5 mil.

22. The multiple layer polymeric film of claim 1 wherein the polyolefin is selected from low density polyethylene, linear low density polyethylene, linear medium density polyethylene, linear ultra-low density polyethylene, high density polyethylene, polypropylene, high density ethylene copolymers, and mixtures of polyolefins.

23. The multiple layer polymeric film of claim 21 wherein at least one layer of polyolefins is present in an amount from about 40% to about 95%.

24. The multiple layer polymeric film of claim 1 wherein the compatibilizer of said second layer is a graft copolymer of polyethylene grafted with maleic anhydride.
